(19) United States
(12) Patent Application Publication
(10) Pub. No.: US 2011/0238023 A1
(43) Pub. Date: Sep. 29, 2011

(54) FEMALE EXTERNAL CATHETER

(76) Inventor: Shamirra Slayton, Macedonia, OH (US)

(21) Appl. No.: 13/072,119

(22) Filed: Mar. 25, 2011

Related U.S. Application Data
(60) Provisional application No. 61/317,342, filed on Mar. 25, 2010.

(51) Int. Cl. A61F 5/455; A61F 13/472

(52) U.S. Cl. 604/329; 604/396

(57) ABSTRACT

A female external catheter device includes a brief having a pair of leg openings that may be opened and closed with lateral straps. A pad assembly is removably connected to the brief. The pad assembly has a funneled portion that is adapted to direct urine away from the vagina. The funnel outlet can be sealed for limited urine collection. This allows the woman on the go to be able to collected urine in the funneled portion and then easily drain the funneled portion by unsealing the outlet of the funneled portion. Alternatively, the woman may elect to remove the entire pad assembly and replace it with a new one. For longer intervals, a urine collection device may be connected to the outlet of the funneled portion.

### CROSS REFERENCE TO RELATED APPLICATIONS

This application claims the benefit of U.S. provisional application 61/317,342 filed Mar. 25, 2010; the disclosure of which is incorporated herein by reference.

### BACKGROUND OF THE INVENTION

1. Technical Field

The invention relates generally to medical devices and, more particularly, to urine collection and removal systems. Specifically, the present invention relates to a urine collection and removal system for a female that functions like an external catheter.

2. Background Information

Most healthcare settings have stuck with the tried and true indwelling catheters. The proper sterile insertion technique requires a nurse to insert the catheter into the urinary meatus and into the bladder. Once inserted into the bladder, the balloon at the end of the insertion tip is then filled with between fifteen to thirty milliliters of sterile water. The filled balloon holds the catheter in place while inserted in the bladder. The indwelling catheter remains in the bladder for approximately one month unless otherwise indicated by the physician. The common practice makes the patient more susceptible to urinary tract infections and a plethora of other infections; especially if the technique or supplies become contaminated. Infections cause doctors to prescribe antibiotic medication which leads to over-prescribing and the over use of antibiotic medication. This is increasingly becoming the cause of the “SuperBug” issues. Bacteria are becoming more and more resistant to antibiotics. Minimizing infections and thus the need for the antibiotics is thus desirable.

Currently female external catheters exist on the market. However, they either require the woman to shave all of her pubic hair allowing an adhesive plastic appendage to be applied to the area with an attached collection drainage bag. There is also an appliance that has to be inserted into the vaginal canal to secure its placement. Both of the above seem to render highly undesired effects.

### SUMMARY OF THE INVENTION

The invention provides a female external catheter. The device is a non-invasive way to collect expelled urine. This device is to be worn like regular women's underwear, yet it includes a uniquely designed unit that funnels urine away from the vagina and into a collection receptacle. This unit is believed to decrease the urethral irritation, urinary tract Infections and other bacterial infections associated with in dwelling Foley catheters.

The invention provides an external catheter device that is unique in that the under garment is aesthetically pleasing to the woman. It may be provided in a multitude of colors. The external catheter helps women maintain their dignity during what can be one of the most embarrassing moments of their lives.

In one configuration, the invention provides an external catheter device combined with a woman's brief. The bottom of the brief defines an opening that accommodates a funneled outlet of a liquid-resistant pad shaped and sized to collect urine as it is expelled from the urethra. An absorbent pad surrounds the at least a portion of the funneled outlet of the pad. Both pads are removable and replaceable.

In one exemplary configuration, the invention provides a comfortable, cotton, aesthetically pleasing disposable brief. This disposable brief may be provided in a variety of colors and sizes to accommodate the complexities of a woman's body. The brief has bilateral Velcro® straps that are composed of stretchy materials to ensure and allow a custom fit for the woman. The vaginal area of the brief is comprised of a soft, cushioned, non-absorbent, non-porous, water repellent material that virtually molds and welcomes the vagina. This material is non-adhering but extremely accommodating to provide the utmost vaginal comfort. The bottom of the brief that houses the vaginal area has a funneled area having a funneled and open bottom which is made of a soft plastic, non-porous material that strategically drains expelled urine and wetness from the urinary meatus/vaginal area out through the bottom opening. The funneled opening easily connects to transparent tubing that has an anti-reflux chamber (doesn't allow urinary back flow). Urine then flows into a urinary collection bag that can either be Velcro® strapped to the thigh or leg region or hung bedside, or a wheelchair etc. For added wetness protection the vaginal area has a cotton adhesive (seal), sanitary pad like, hyper absorbent insert that outlines the vaginal and funneled drain. This adhesive sanitary pad like can be changed or removed as needed without having to discard the entire brief each time wetness or leakage occurs.

Another configuration of the invention provides a brief having an external catheter wherein the outlet of the funneled pad can be sealed with a closure device integral to the pad or a removable cap. The closure device allows for short term urine collection. In this configuration, the funneled pad functions as a urine collection receptacle. This allows the woman on the go to be able to uncap the funneled pad and empty any collected urine over a toilet or other urinary collection receptacle. Alternatively, the user may remove the removable insert which includes the funneled pad, discard it, and then assemble a new pad assembly. This configuration also can be used with the drainage tubing which is then connected to a collection device permitting longer intervals of usage between emptying and changing of the removable insert.

### BRIEF DESCRIPTION OF THE DRAWINGS

FIG. 1 is a front perspective view of the female external catheter with the tubing and collection bag removed.

FIG. 2 is a front elevation view of FIG. 1.

FIG. 3 is a top plan view of FIG. 1.

FIG. 4 is a perspective view of the female external catheter assembled in accordance with one exemplary configuration of the invention.

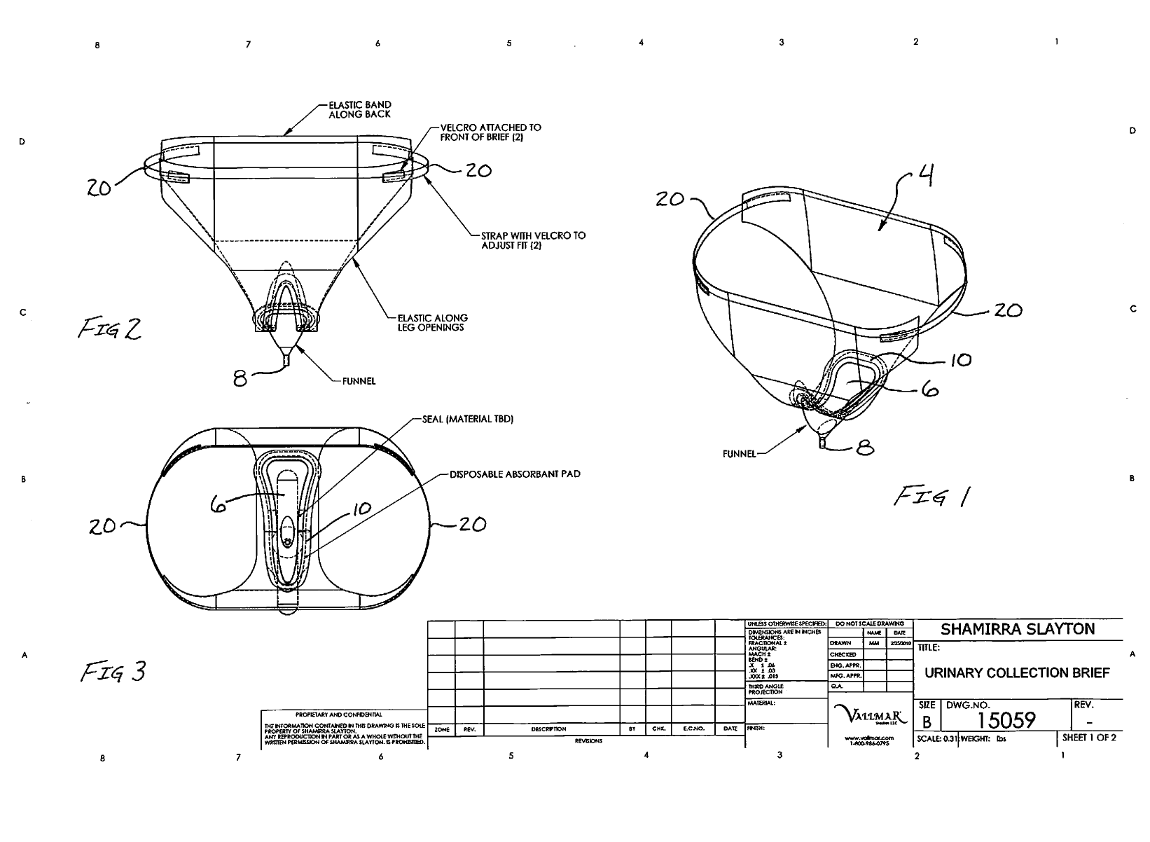

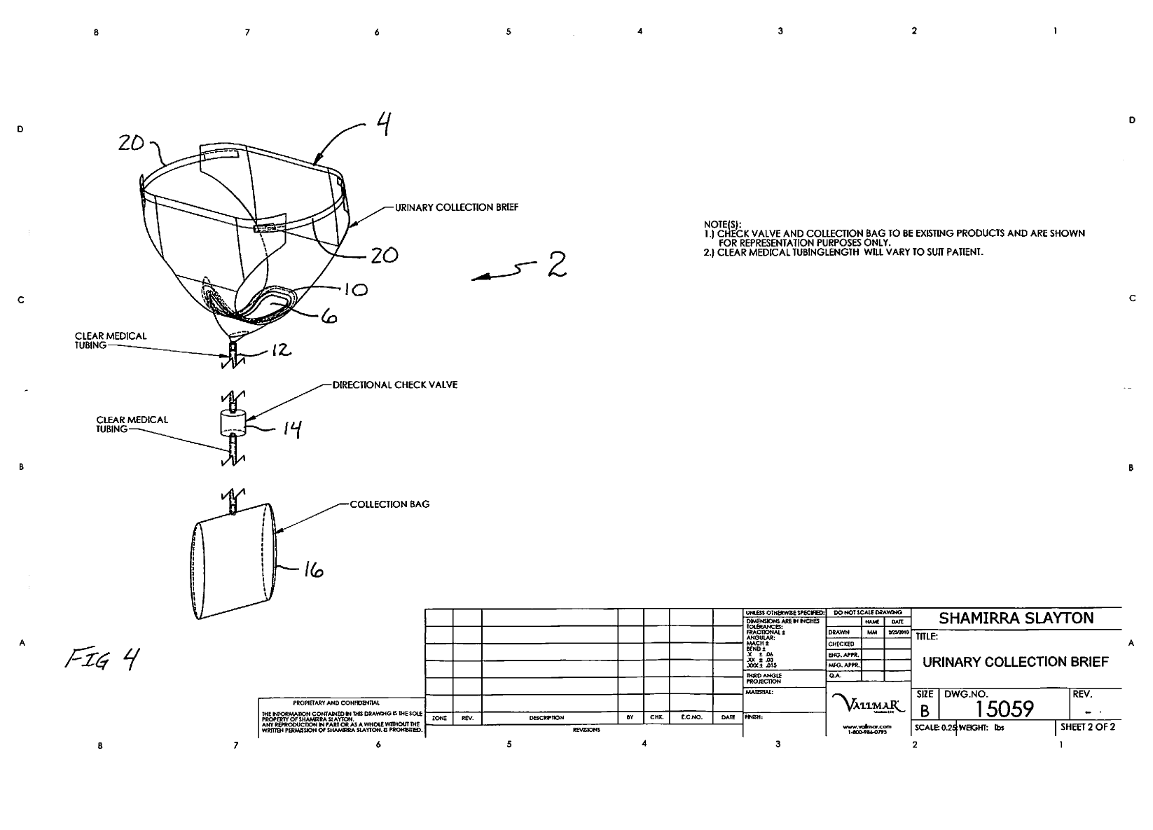

Similar numbers refer to similar parts throughout the specification.

### DETAILED DESCRIPTION OF THE INVENTION

An exemplary configuration of an external female catheter device of the invention is indicated generally by the numeral 2 in the accompanying drawings. Device 2 generally includes a brief 4, a liquid-resistant pad 6 having a funneled portion that defines an outlet 8, and an absorbent outer pad 10 disposed about at least a portion of the funneled portion of liquid-resistant pad 6. In one configuration, catheter 2 also includes tubing 12, a valve 14, and a collection device 16. Pads 6 and 10 may be separately removable from brief 4 or they may be joined together as an assembly that is commonly removable and replaceable with respect to brief 4. Device 2 may be worn for a variety of reasons such as, for example, bladder incontinence due to age or medication, over active bladder, bladder weakness, bladder spasms or just for precautionary measures.

Brief 4 may be a in the form of a typical women's undergarment provided in different size, shapes, and materials for different women. The material may be cotton, polyester, microfiber, Rayon, Spandex, silk, a fabric blend, or other soft flexible material used to fabricate undergarments. Brief 4 may include lateral straps 20 that allow brief 4 to be removed without the need to slide brief 4 down over the knees and feet. One end of each lateral strap 20 may be secured to one portion of brief 4 with the other end of lateral strap 20 is configured to be releasably and rejoinably connected to another portion of brief 4. A connector that provides for repeated attachments and repeated detachments is used. A hook and loop connector such as that sold under the Velcro® may be used. In other configurations, snaps, buttons, ties, buckles, or other connectors may be used.

Brief 4 has a unique design with specialized contouring that caters to a woman's body. Pads 6 and 10 are positioned at the bottom area of brief 4 and the particular user may adjust the location of pads 6 and 10 when the woman installs pads 6 and 10. The bottom area of brief 4 defines an opening through which the funneled portion of pad 6 projects. The opening defined by brief 4 may be larger than the funnel portion thus allowing the woman to select a position for pad 6 with respect to brief 4 as desired. The particular location of pad 10 also may be selected by the user.

In one configuration, the bottom area of brief 4 defines openings or perforations beginning at the top of the vagina area extending to the buttock area. These openings or perforations enable pads 6 and 10 to be secured to brief 4 to prevent undesired movement of pads 6 and 10 with respect to brief 4. In this configuration of device 2, pads 6 and 10 are removably connected to brief 4 with a plurality of short pad straps that are looped through the openings or perforations. Connectors such as hook and loop connectors are used to secure the pad straps in place. In another configuration, pads 6 and 10 may be secured directly to brief 4 with hook-and-loop connectors disposed directly between brief 4 and at least one of pads 6 and 10. Pads 6 and 10 also may be removably and reattachably secured with adhesive. For example, the replaceable pads 6 and 10 may include adhesive strips that extend around all or a portion of pads 6 and/or 10. The adhesive strips may be provided with release covers that are peeled away and disposed when pads 6 and 10 are installed. Other connectors also may be used that allow pads 6 and 10 to be removed and replaced. When the entire device 2 is disposable, then pads 6 and 10 may be secured via stitching, adhesives, or integrally formed with brief 4.

Pad 10 may be a cotton pad (outer parameters) or other absorbent pad disposed about the funneled portion of pad 6 or about the outer perimeter or a portion of the outer perimeter of pad 6. Pad 6 is liquid resistant and defines a funneled portion configured to direct urine to outlet 8. Pad 6 may be a cushioned, water repellent, padded hypoallergenic structure (where the vagina sits in comfort). Pad 6 may be a plastic or rubber material or a coated flexible material. The center of pad 6 has two parallel sloped areas connected to a funneled area (that strategically drains urine away from body).

Outlet 8 of the funneled portion of pad 6 can be sealed with a closure device such as one that is integral to pad 6, a devices that pinches outlet 8 closed, or a removable cap. The closure device allows the funneled portion to be sealed for short term or interval urine collection. In this configuration, the funneled pad 6 functions as a urine collection receptacle. This allows the woman on the go to be able to unseal the funneled pad 6 and empty any collected urine over a toilet or other urinary collection receptacle. The woman also may remove the removable insert (pads 6 and 10) which includes the funneled pad, discard the insert, and then assemble a new pad assembly (pads 6 and 10). This configuration also can be used with the drainage tubing which is then connected to a collection device permitting longer intervals of usage between emptying and changing of the removable insert.

An anti-reflux chamber or valve 14 may be integrally formed with outlet 8 or provided in tubing 12 to prevent urinary back flow. Valve 14 or tubing 12 may be connected to a collection device 16. Collection device 16 may be carried by the user's leg with straps allowing the user to be mobile or a collection drainage bag 16 that usually hangs bedside for the immobile consumer.

Leg drainage urine collection bag 16 may be composed of plastic, vinyl or various hypoallergenic materials. This bag 16 is usually attached to the leg or thigh of a mobile consumer. Larger capacity urine collection drainage bag 16 may be composed of plastic, vinyl or various hypoallergenic materials. The larger capacity bag 16 may be attached to the bed or wheelchair for immobile consumers.

Device 2 is believed to have the advantage of decreasing the incidence of urinary tract/hospital acquired infections associated with indwelling catheters and improper insertion methods. Device 2 is also believed to have the advantage of decreasing the incidence of possible rupture to the urinary meatus due to pulling and pressure during mobility. Device 2 should also decrease skin breakdown due to the device being changed daily and decrease incidence of antibiotic overexposure associated with catheter changes. Other advantages include no hospitalization required for application or removal, device 2 can be changed daily by either the healthcare provider or the wearer. Device 2 is easy to apply and can be used in a variety of healthcare settings or home. Device 2 is also affordable and maintains consumer dignity. The entire device 2 is disposable.

In the foregoing description, certain terms have been used for brevity, clearness, and understanding. No unnecessary limitations are to be implied therefrom beyond the requirement of the prior art because such terms are used for descriptive purposes and are intended to be broadly construed. Moreover, the description and illustration of the invention is an example and the invention is not limited to the exact details shown or described. Throughout the description and claims of this specification the words “comprise” and “include” as well as variations of those words, such as “comprises,” “includes,” “comprising,” and “including” are not intended to exclude additives, components, integers, or steps.

1. A female external catheter device comprising:
a brief having a pair of leg openings with a bottom portion disposed between the leg openings;
the bottom portion defining a pad opening disposed between the leg openings;
a pad assembly removably connected to the brief at the pad opening;
the pad assembly having an inner pad that is substantially liquid resistant; the inner pad having an outlet and an outer perimeter; and
the pad assembly having an outer pad formed from an absorbent material disposed about a portion of the inner pad.

2. The device of claim 1, further comprising a valve connected to the outlet.

3. The device of claim 2, further comprising a collection device connected to the valve.

4. The device of claim 1, wherein the inner pad defines sloped sides directed toward the outlet to define a funneled portion; the funnel portion having an inlet adapted to receive urine.

5. The device of claim 4, wherein the outer pad is disposed around at least a portion of the inlet to the funneled portion.

6. The device of claim 1, wherein the pad assembly is removably connected to the brief with adhesive.

7. The device of claim 1, wherein the pad assembly is removably connected to the brief with pad straps.

8. The device of claim 1, wherein the outer pad completely surrounds the outer perimeter of the inner pad.

9. The device of claim 1, wherein the leg openings of the brief are at least partially defined by lateral straps that are attachable to and detachable from other portions of the brief to allow the brief to be removed from the user's body without sliding it down over the user's knees and feet.

10. The device of claim 9, wherein the lateral straps are attachable to and detachable from other portions of the brief with hook and loop fasteners.

11. A female external catheter device comprising:
a brief having a pair of leg openings and a pad opening disposed between the leg openings;
a pad assembly removably connected to the brief at the pad opening; and
the pad assembly having a funneled portion having an inlet and an outlet; the funneled portion adapted to direct urine away from the vagina.

12. The device of claim 11, wherein the leg openings of the brief are at least partially defined by lateral straps that are attachable to and detachable from other portions of the brief to allow the brief to be removed from the user's body without sliding it down over the user's knees and feet.

13. The device of claim 12, wherein the lateral straps are attachable to and detachable from other portions of the brief with hook and loop fasteners.

14. The device of claim 11, further comprising a valve connected to the outlet.

15. The device of claim 14, further comprising a collection device connected to the valve.

16. The device of claim 11, wherein the pad assembly includes an inner pad and an outer pad; the inner pad defining the funneled portion; the inner pad being liquid-resistant; the outer pad being absorbent; and the outer pad being disposed around at least a portion of the inlet to the funneled portion.

17. The device of claim 16, wherein the pad assembly is removably connected to the brief with adhesive.

18. The device of claim 11, further comprising a closure device that seals the outlet to the funneled portion.

19. The device of claim 11, wherein the pad assembly is removably connected to the brief with pad straps.
